(19) United States
(12) Patent Application Publication
(10) Pub. No.: US 2026/0097093 A1
(43) Pub. Date: Apr. 9, 2026

(54) TARGETED AMATOXIN CONJUGATE FOR THE TREATMENT OF SOLID TUMORS

(71) Applicant: Heidelberg Pharma Research GmbH, Ladenburg (DE)

(72) Inventors: Christoph Müller, Ladenburg (DE); Werner Simon, Ladenburg (DE); Christian Lutz, Ladenburg (DE); Susanne Werner-Simon, Ladenburg (DE); Torsten Hechler, Ladenburg (DE); Michael Kulke, Ladenburg (DE); Andreas Pahl, Ladenburg (DE)

(73) Assignee: Heidelberg Pharma Research GmbH, Ladenburg (DE)

(21) Appl. No.: 19/350,762

(22) Filed: Oct. 6, 2025

Related U.S. Application Data
(63) Continuation of application No. 17/046,497, now Pat. No. 12,544,419.

(30) Foreign Application Priority Data
Apr. 13, 2018 (EP) 18167265.0

(51) Int. Cl. A61K 38/12; A61K 47/65; A61K 47/68; A61P 35/00

(52) U.S. Cl. CPC A61K 38/12; A61K 47/65; A61K 47/6831; A61K 47/6871; A61P 35/00

(57) ABSTRACT

An amatoxin-linker construct contains an amatoxin according to formula (I)

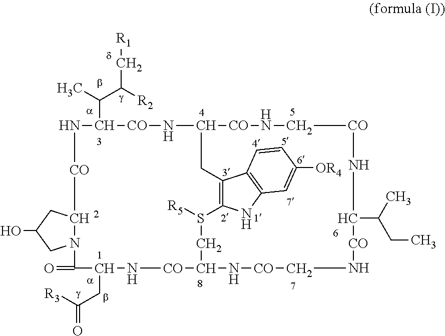

wherein R1 and R2 are each —OH, R3 is NH2, or a linker which carries a reactive group Y for linking said amatoxin to a target-binding moiety, R4 is H or a linker which carries a reactive group Y for linking said amatoxin to a target-binding moiety, R5 is absent or ═O, and wherein R3 and R4 cannot be the same. The amatoxin-linker construct is used in the manufacture of a binding moiety-toxin conjugate for the treatment of a solid tumor, and a respective binding moiety-toxin conjugate can be used for the treatment of a solid tumor.

### CROSS-REFERENCE TO RELATED APPLICATIONS

This present application is a continuation of U.S. application Ser. No. 17/046,497, filed on Oct. 9, 2020, which is the National Stage entry under § 371 of International Patent Application No. PCT/EP2019/059516, filed on Apr. 12, 2019, and which claims the benefit of European Patent Application No. 18167265.0, filed on Apr. 13, 2018, which are incorporated by reference in their entireties herein.

### REFERENCE TO A SEQUENCE LISTING

The present application is accompanied by an XML file as a computer readable form containing the sequence listing entitled, “2025-09-29-SeqList-006208USCONT01-as-filed”, created on Sep. 29, 2025, with a file size of 17,464 bytes, the content of which is hereby incorporated by reference in its entirety.

### BACKGROUND OF THE INVENTION

### Field of the Invention

The present application relates to targeted amatoxin conjugates for the treatment of solid tumors.

### Description of Related Art

The treatment of solid tumors is an ongoing challenge, with little advancements over the last decades. While quite a few new treatment modalities have been developed recently, there is still a need for further improvements, to increase survival rates of patients suffering from solid tumors, and improve their quality of life. One promising treatment modality are antibody drug conjugates, where a highly toxic entity that has the capacity of killing cells is coupled to a target binding entity that shuttles the toxin to a site of disease.

The applicant has, in the past developed so called ATACs, i.e., Antibody Targeted Amanitin Conjugates, where an amanitin toxin is coupled to a highly target specific antibody. Amanitins are bicyclic peptides of eight amino acids that occur, naturally, in several species of the Amanita genus of mushrooms, one being the death cap (Amanita phalloides). Amanitins have the following general structure:

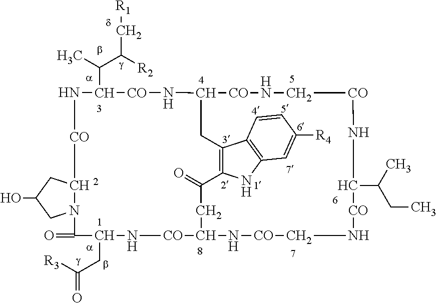

| | R₁ | R₂ | R₃ | R₄ |
|---|---|---|---|---|
| α-amanitin | OH | OH | NH₂ | OH |
| β-amanitin | OH | OH | OH | OH |
| γ-amanitin | H | OH | NH₂ | OH |
| ε-amanitin | H | OH | OH | OH |
| amanin | OH | OH | OH | H |
| amaninamide | OH | OH | NH₂ | H |
| amanullin | H | H | NH₂ | OH |
| amanullinic acid | H | H | OH | OH |

Amatoxins bind to RNA polymerase II with very high affinity resulting in a dramatic decrease in protein synthesis and ultimately in apoptosis in both dividing and resting cells (Cochet-Meilhac and Chambon 1974; Cochet-Meilhac et al. 1974). Alpha-amanitin only slightly binds to RNA polymerase III and shows no binding to RNA polymerase I. When bound to RNA polymerase II, amanitins inhibit the translocation of the enzyme on the RNA and DNA and thus transcription rate is slowed down by over 1000 fold finally resulting in apoptosis of the cell.

One ATAC that is currently under development is called HDP 101, and is disclosed in PCT application PCT/EP2017/084431. It comprises an amatoxin with an amino acid 4 with a 6′-deoxy position (═R4 in the above figure); and an amino acid 8 with a S-deoxy position. The linker is a PAB Val Ala Linker.

This amatoxin is conjugated, via amino acid 1 and a cleavable linker conjugated thereto, to an anti BCMA-binding antibody called J22.9—ISY. The structure of the amatoxin linker conjugate used in HDP101 is called 30.2115, and shown in a table herein below.

This ATAC has shown very good efficacy in the treatment of hematologic malignancies, in particular of lymphoma. Such hematologic malignancies are called herein “liquid tumors” as well.

However, the applicants have realized that there is still room for improvement in the treatment of solid tumors, which represent a more challenging target for ATACa than liquid tumors.

It is hence one object of the present invention to provide new treatment options of solid tumors. It is one further object of the present invention to improve the efficacy of ATACs in solid tumors.

These and further objects are met with methods and means according to the independent embodiments of the present invention. The dependent embodiments are related to specific embodiments.

### SUMMARY OF THE INVENTION

The present invention provides targeted amatoxin conjugates for the treatment of solid tumors. The invention and general advantages of its features will be discussed in detail below.

The invention includes but is not limited to the following embodiments:
1. A method of treating a patient afflicted with a solid tumor, the method comprising: administering to said patient an effective amount of a binding moiety-toxin conjugate,
wherein the binding moiety-toxin conjugate comprises an amatoxin-linker construct selected from the group consisting of:

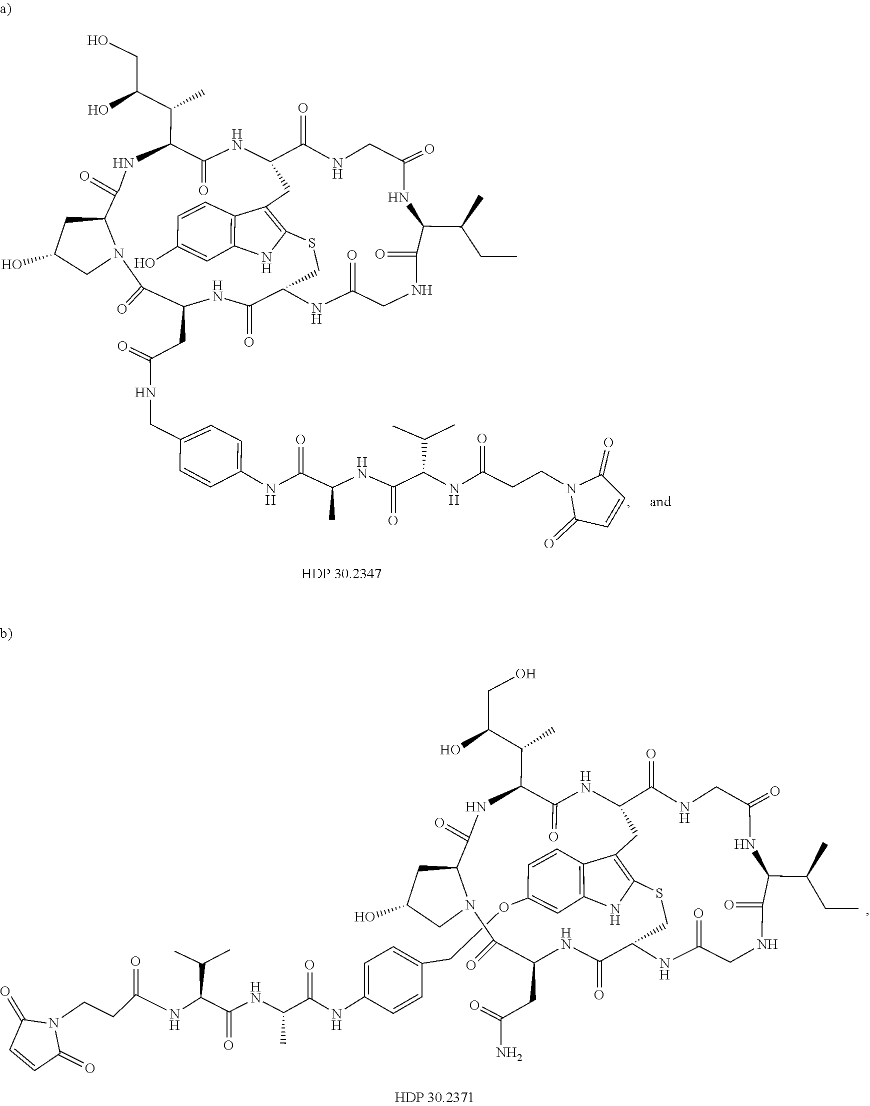

and
wherein said solid tumor is resistant to a binding moiety-toxin conjugate whose amatoxin according to formula (I) does not comprise an —OR₄ substituent at 6′-position of the indol, wherein formula (I) is defined as

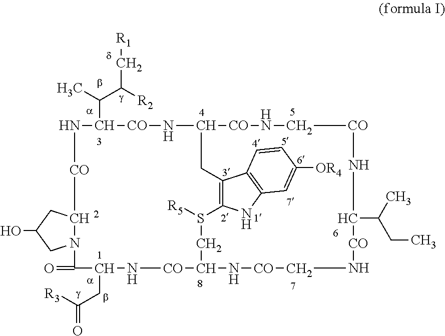

and wherein
R1 and R2 are each —OH,
R3 is NH2, or a linker which carries a reactive group Y for linking said amatoxin to a target-binding moiety,
R4 is H or a linker which carries a reactive group Y for linking said amatoxin to a target-binding moiety,
R5 is absent or ═O,
wherein R3 and R4 cannot be the same, and
wherein the target-binding moiety is an antibody, antibody fragment, antibody-based binding protein, or an antibody mimetic, all of which retain target binding properties.
2. The method according to embodiment 1, wherein said target-binding moiety binds at least one target selected from the group consisting of Her2, and PSMA.
3. The method according to embodiment 1, wherein the solid tumor is at least one selected from the group consisting of (a) sarcoma, (b) blastoma, and (c) carcinoma.
4. The method according to embodiment 1, wherein said target-binding moiety comprises an antibody or fragment or a derivative thereof which comprises an engineered cysteine residue.
5. The method according to embodiment 4, wherein said cysteine residue is selected from the group consisting of heavy chain 118Cys, heavy chain 239Cys, and heavy chain 265Cys, according to the EU numbering system.

### BRIEF DESCRIPTION OF THE DRAWINGS

The following list gives an overview of the Figures and their content:

FIG. 1A shows the results of a cell viability assay used with JIMT-1 cells.

FIG. 1B shows the results of a cell viability assay used with JIMT-1 cells.

FIG. 2A shows the results of growth inhibition experiments carried out with tumor xenografts of JIMT cells.

FIG. 2B shows the results of growth inhibition experiments carried out with tumor xenografts of JIMT cells.

FIG. 3 shows the results of a cell viability assay used with LNCaP cells.

FIG. 4A shows the results of growth inhibition experiments carried out with tumor xenografts of LnCap cells.

FIG. 4B shows the results of growth inhibition experiments carried out with tumor xenografts of LnCap cells.

FIG. 4C shows the results of growth inhibition experiments carried out with tumor xenografts of LnCap cells.

FIG. 5 shows the results of a cell viability assays used with Raji cells.

FIG. 6A shows the results of survival experiments that were carried out with i.v. injected Raji Luc cells.

FIG. 6B shows the results of Survival experiments that were carried out with i.v. injected NALM6 cells.

FIG. 7A shows the results of growth inhibition experiments that were carried out with tumor xenografts of Raji cells injected into mice s.c.

FIG. 7B shows the results of growth inhibition experiments that were carried out with tumor xenografts of Raji cells injected into mice s.c.

### DETAILED DESCRIPTION OF THE INVENTION

Before the invention is described in detail, it is to be understood that this invention is not limited to the particular component parts of the devices described or process steps of the methods described as such devices and methods may vary. It is also to be understood that the terminology used herein is for purposes of describing particular embodiments only, and is not intended to be limiting. It must be noted that, as used in the specification and the appended embodiments, the singular forms “a”, “an”, and “the” include singular and/or plural referents unless the context clearly dictates otherwise. It is moreover to be understood that, in case parameter ranges are given which are delimited by numeric values, the ranges are deemed to include these limitation values.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

Furthermore, the content of the prior art documents referred to herein is incorporated by reference. This refers, particularly, for prior art documents that disclose standard or routine methods. In that case, the incorporation by reference has mainly the purpose to provide sufficient enabling disclosure, and avoid lengthy repetitions.

According to a first aspect, an amatoxin-linker construct comprising an amatoxin according to formula (I)

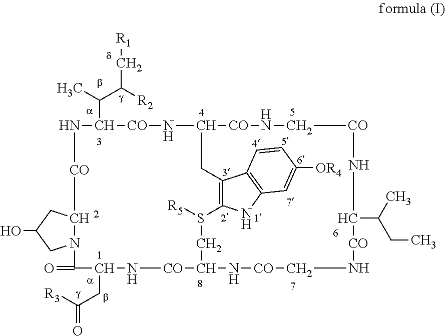

is provided, wherein:
R1 and R2 are each —OH
R3 is NH2, or a linker which carries a reactive group Y for linking said amatoxin to a target-binding moiety,
R4 is H or a linker which carries a reactive group Y for linking said amatoxin to a target-binding moiety,
R5 is absent or ═O,
wherein R3 and R4 cannot be the same.

Said construct is provided for use in the manufacture of a binding moiety-toxin conjugate for the treatment of a solid tumor.

One of the key features of these constructs is the —OR4 substituent on position 4 in the indole ring of amino acid 4 (Trp) of the molecule.

The inventors have surprisingly shown that binding moiety-toxin conjugates comprising such amatoxin linker constructs have superior potency against solid tumors, or cells comprised therein. This is especially true when compared to structurally similar binding moiety-toxin conjugates which do not have the —OR4 on position 4, but have a hydrogen substituent instead.

Interestingly, no such obvious difference can be observed in the treatment of liquid tumors, were both variants have a comparable potency, as disclosed in PCT application PCT/EP2017/084431.

The term “solid tumor”, as used herein, relates to all cancer types affecting body tissue or organs, with the exception of the blood, bone marrow and lymphatic system.

The term “liquid tumor”, as used herein, relates to any cancer or malignancy that occurs in the fluid of a patient's body, for instance blood, bone marrow or lymph, including leukemia, lymphoma and myeloma.

It is important to understand that —OR4 can either be —OH, or —O-linker. In particular, if the linker is a cleavable linker or a self immolative linker, the linker will degrade over time, and the final step of immolation will result in the hydrolysis of the —O-linker bond, hence releasing an —OH. For this reason, the cleavage/immolation will deliver a 6-OH-Trp.

According to one embodiment, the linker is a cleavable linker and/or a self immolative linker.

The term “cleavable linker”, as used herein, relates to a linker that is typically susceptible to cleavage under intracellular conditions. Suitable cleavable linkers include, for example, a peptide linker cleavable by an intracellular protease, such as lysosomal protease or an endosomal protease. In exemplary embodiments, the linker can be a dipeptide linker, such as a valine-citrulline (val-cit), a valine-alanine (val-ala), a phenylalanine-lysine (phe-lys) linker, or maleimidocapronic “valine-citruline-p-aminobenzyloxycarbonyl (mc-Val-Cit-PABA) linker. Such linkers are e.g. disclosed in U.S. Pat. No. 6,214,345.

Another linker is Sulfosuccinimidyl-44N-maleimidomethyl]cyclohexane-1-carboxylate (smcc). Sulfo-smcc conjugation occurs via a maleimide group which reacts with sulfhydryls (thiols, —SH), while its Sulfo-NHS ester is reactive toward primary amines (as found in Lysine and the protein or peptide N-terminus). Yet another linker is maleimidocaproyl (mc). Other suitable linkers include linkers hydrolyzable at a specific pH or a pH range, such as a hydrazone linker. Additional suitable cleavable linkers include disulfide linkers. The linker may be covalently bound to the antibody to such an extent that the antibody must be degraded intracellularly in order for the drug to be released e.g. the me linker and the like. Other pH sensitive cleavable linkers that comprise a cyclic benzylidene acetal linker are disclosed in EP3130587.

The term “self immolative linker”, as used herein, relates to a linker which degrades spontaneously after an initial reaction has taken place. One example of a self-immolative linker is p-aminobenzyl alcohol (PAB), which is often conjugated to a cleavable dipeptide, like valine-citrulline (val-cit), or valine-alanine (val-ala), via the aromatic amine group and via a carbamate group to a primary or secondary amine of the toxin. Cleavage of the dipeptide by a suitable protease triggers a 1,6-elimination of PAB and carbon dioxide in PAB and concomitant release of the free toxin. The p-aminobenzyl moiety can also be coupled directly by alkylation to an amino-, hydroxy- or phenol-group of the toxin. Cleavage of the dipeptide results then by 1,6-elimination to fragmentation of the benzylamine or benzylether bond with concomitant release of the toxin.

Other protease triggers for this PAB based self-immolative systems are exemplified but not limited to, by the glycine-proline-leucine-glycine (gly-pro-leu-gly) motive, cleavable by matrix metalloproteases (MMP II) or the alanine-alanine-proline-valine (ala-ala-pro-val) motive, cleavable by elastase.

Another example of self-immolative linker is represented by beta-glycosides of p-hydroxybenzyl alcohol with glucuronic acid. Toxins can be conjugated to the benzylic carbon atom by the above-mentioned groups and a target binding moiety via the meta-position of the aromatic ring. Upon cleavage of the glyosidic bond by beta-glucuronidase the toxin is liberated by an 1,6-elimination process.

According to one embodiment, the linker is cleavable by at least one agent selected from the group consisting of
Cysteine protease
Metallo protease
Serine protease
Threonine protease
Aspartic protease

Cysteine proteases, also known as thiol proteases, are enzymes that degrade proteins. These proteases share a common catalytic mechanism that involves a nucleophilic cysteine thiol in a catalytic triad or dyad. Cysteine proteases are commonly encountered in fruits including the papaya, pineapple, fig and kiwifruit

The MEROPS protease classification system has Cysteine proteases under C. Metalloproteases are protease enzymes whose catalytic mechanism involves a metal. Most metalloproteases require zinc, but some use cobalt. The metal ion is coordinated to the protein via three ligands. The ligands co-ordinating the metal ion can vary with histidine, glutamate, aspartate, lysine, and arginine. The fourth coordination position is taken up by a labile water molecule.

Serine proteases are enzymes that cleave peptide bonds in proteins, in which serine serves as the nucleophilic amino acid at the (enzyme's) active site. They are found ubiquitously in both eukaryotes and prokaryotes. Serine proteases fall into two broad categories based on their structure: chymotrypsin-like (trypsin-like) or subtilisin-like.

The MEROPS protease classification system has Cysteine proteases under S.

Threonine proteases are a family of proteolytic enzymes harbouring a threonine (Thr) residue within the active site. The prototype members of this class of enzymes are the catalytic subunits of the proteasome, however the acyltransferases convergently evolved the same active site geometry and mechanism.

Aspartic proteases are a catalytic type of protease enzymes that use an activated water molecule bound to one or more aspartate residues for catalysis of their peptide substrates. In general, they have two highly conserved aspartates in the active site and are optimally active at acidic pH. Nearly all known aspartyl proteases are inhibited by pepstatin. The MEROPS protease classification system has Cysteine proteases under A.

In particular embodiments the linker is cleavable by at least one agent selected from the group consisting of
Cathepsin A or B
Matrix metalloproteinases (MMPs)
Elastase
Beta-glucuronidase
Beta-galactosidase

According to one further embodiment, the linker the linker comprises a motif selected from the group consisting of

Val Ala
Val Cit
Val Lys
Val Arg
(SEQ ID NO: 11)
Phe Lys Gly Pro Leu Gly
(SEQ ID NO: 12)
Ala Ala Pro Val
Beta-glucuronide
Beta-galactoside

According to one further embodiment, the reactive group Y in the linker is at least one selected from the group consisting of:

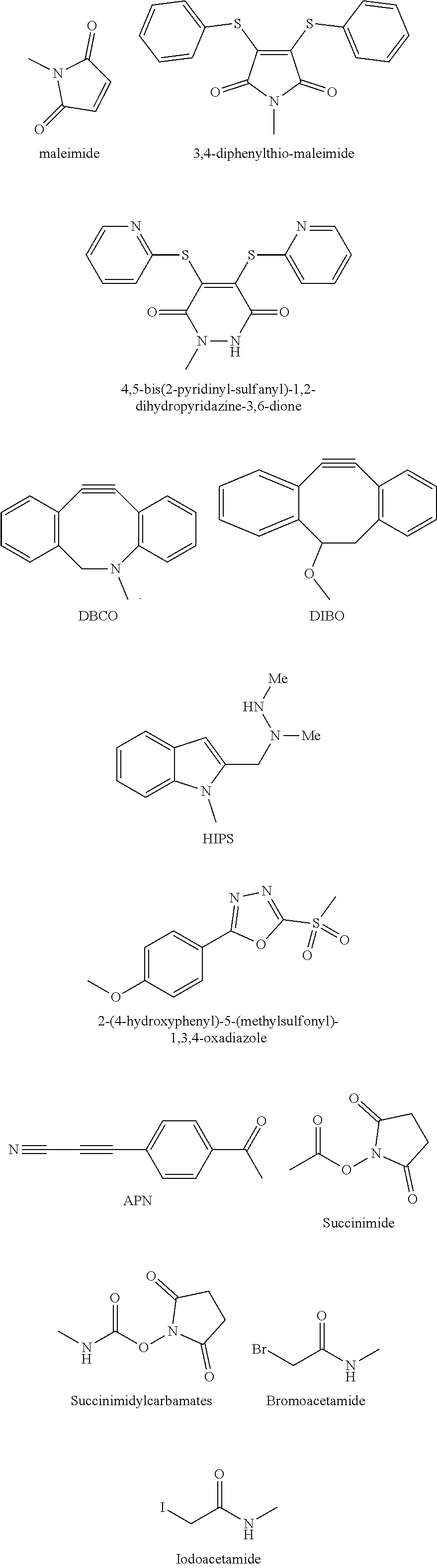

According to one further embodiment, the linker comprises a para Aminobenzoyl Val Ala maleimidopropyl motif

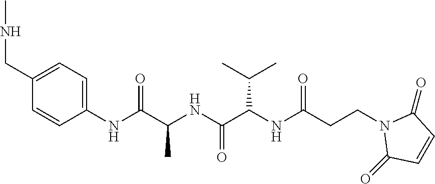

According to one further embodiment, the linker in the amatoxin-linker construct is a non-cleavable linker. The terms “stable linker” and “non-cleavable linker” are used interchangeably herein.

Typically, non-cleavable linkers release the drug after the binding moiety, i.e., the monoclonal antibody to which the toxin linker construct is conjugated, is degraded intracellularly, e.g., in the lysosomes. A typical non cleavable linker is the maleimide alkane linker:

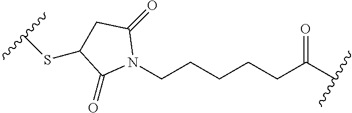 [SMILES: *SC1CC(=O)N(CCCCCC(*)=O)C1=O]

Another typical non-cleavable linker is the SMCC linker as used in Ado-trastuzumab Emtansine (T-DM1)

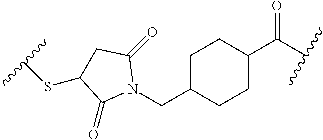 [SMILES: *SC1CC(=O)N(CC2CCC(C(*)=O)CC2)C1=O]

In one embodiment, R3 is not a non-cleavable linker. In another embodiment, R3 is a cleavable linker, preferably a self immolative linker. In another embodiment, R4 is a non cleavable, a cleavable or a self immolative linker.

According to one further embodiment, the amatoxin-linker construct has a formula selected from the group consisting of:

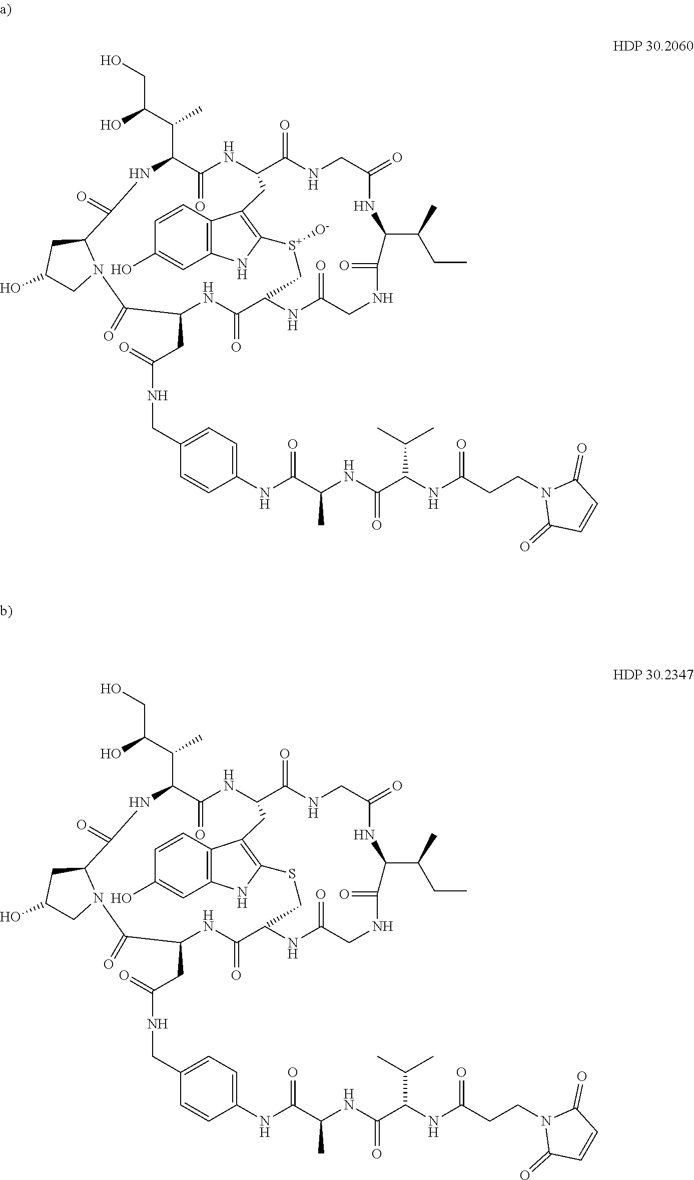

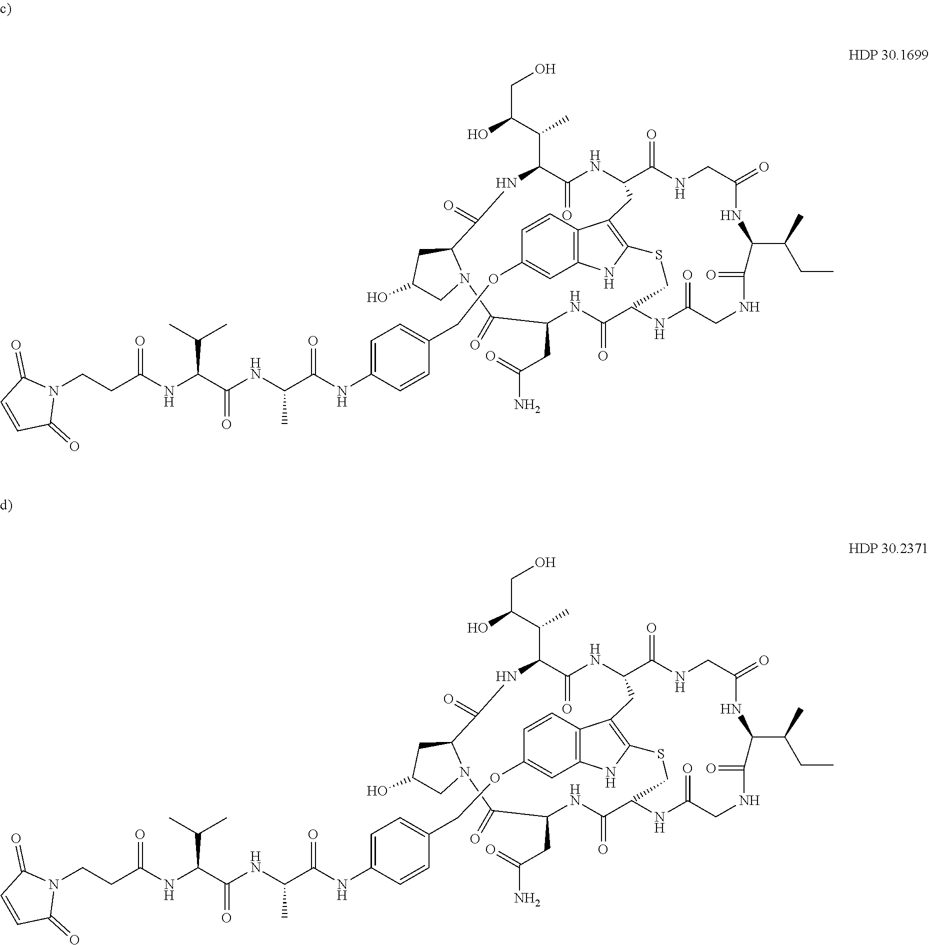

TABLE 1
Some molecular characteristics of these four amatoxin-linker constructs.

| Applicant's | | | | | | |
|---|---|---|---|---|---|---|
| identifier | Trp | Sulfur bridge | Linker at | R4 | R3 | Linker |
| 30.1699 | 6-Hyd-Trp | sulfoxide | aa4 | Linker | H | p-Amino-Benzyl |
| 30.2371 | 6-Hyd-Trp | thioether | aa4 | Linker | H | Ala Val |
| 30.2060 | 6-Hyd-Trp | sulfoxide | aa1 | H | Linker | maleimidopropyl |
| 30.2347 | 6-Hyd-Trp | thioether | aa1 | H | Linker | |

According to one further embodiment, the amatoxin-linker construct which is provided for use in the manufacture of a binding moiety-toxin conjugate for the treatment of a solid tumor, wherein said solid tumor is resistant to a binding moiety-toxin conjugate whose amatoxin does not comprise an —OR4 substituent at 6′-position of the indol.

According to another aspect of the invention, a binding moiety-toxin conjugate is provided, said conjugate for use in the treatment of a human or animal subject being
(i) diagnosed for,
(ii) suffering from or (iii) being at risk of developing a solid tumor, which conjugate comprises
   (a) the amatoxin-linker construct of the above description, and
   (b) a target-binding moiety; and
wherein the linker links said amatoxin and said target-binding moiety.

According to another aspect of the invention, such binding moiety-toxin conjugate is provided for use in manufacture of a medicament for the treatment of a human or animal subject being
(i) diagnosed for, (ii) suffering from or (iii) being at risk of developing a solid tumor.

According to another aspect of the invention, a method for treating or preventing a solid tumor is provided, said method comprising administering to a subject in need thereof an effective amount of such binding moiety-toxin conjugate.

According to one embodiment of the invention, the target-binding moiety is an antibody, antibody fragment, antibody-based binding protein, or an antibody mimetic, all of which retain target binding properties.

An “antibody”, as used herein, is preferably a monoclonal antibody. As used herein, the term “monoclonal antibody (mAb)”, shall refer to an antibody composition having a homogenous antibody population, i.e., a homogeneous population consisting of a whole immunoglobulin, or a fragment or derivative thereof retaining target binding capacities. Particularly preferred, such antibody is selected from the group consisting of IgG, IgD, IgE, IgA and/or IgM, or a fragment or derivative thereof retaining target binding capacities.

As used herein, the term “antibody fragment” shall refer to fragments of such antibody retaining target binding capacities, e.g.
a CDR (complementarity determining region)
a hypervariable region,
a variable domain (Fv)
an IgG heavy chain (consisting of VH, CH1, hinge, CH2 and CH3 regions)
an IgG light chain (consisting of VL and CL regions), and/or
a Fab and/or F(ab)2.

Other embodiments encompass Camelid Antibodies, Shark antibodies, heavy chain portion of a Fab(Fd) fragment, which consists of the VH and CH1 domains; a variable fragment (Fv) fragment; a domain antibody (dAb) fragment, which comprises a single variable domain, a single chain FvFragment (scFv); (viii) a diabody; a linear antibody, which comprises a pair of tandem Fvsegments (VH-CH1-VH-CH1) which, together with complementarity light chain polypeptides, form a pair of antigen binding regions; and other non-full length portions of immunoglobulin heavy and/or light chains, or mutants, variants, or derivatives thereof, alone or in any combination An antibody based binding protein as used herein comprises at least one antibody-derived VH, VL, or CH immunoglobulin domain in the context of other non-immunoglobulin, or non-antibody derived components.

An antibody mimetic, as used herein, is an organic compound that, like antibodies, can specifically bind antigens, but is not structurally related to antibodies. This definition encompasses, for example, Ankyrin Repeat Proteins, C-Type Lectins, A-domain proteins of S. aureus, Transferrins, Lipocalins, 10th type III domains of fibronectin, Kunitz domain protease inhibitors, Ubiquitin derived binders, Gamma Crystallin derived binders, Cysteine knots or knottins, thioredoxin A scaffold based binders, nucleic acid aptamers, Artificial Antibodies produced by molecular imprinting of polymers, Peptide libraries from bacterial genomes, SH-3 domains, Stradobodies, “A domains” of membrane receptors stabilised by disulfide bonds and Ca2+, CTLA4-based compounds, Fyn SH3, and Aptamers.

According to one embodiment, said target-binding moiety binds at least one target selected from the group consisting of
Her2, and/or
PSMA.

According to one embodiment of the invention, said target-binding moiety is at least one antibody selected from the group consisting of
Trastuzumab, and/or
h3/F11.

The inventors saw that tumor cells, such as Raji or Raji-Luc, grow, when applied subcutaneously, as homogeneous solid tumors with defined structure and connective tissue. These subcutaneously implanted tumor cells are indicative for solid tumors since important properties of solid tumors are given such as penetration hindrance and diffusion hindrance. Besides, subcutaneous models in general are prerequisites for the preclinical development of new drugs intended for the treatment of cancer.

When injected intravenously, however, Raji or Raji-Luc develop the phenotype of a liquid tumor, e.g., a leukemia. For this reason, Raji or Raji-Luc are a suitable model to show the different effects of the described toxin linker conjugates in treatment of solid vs liquid tumors.

The CDR sequences of antibody h3/F11 are disclosed in EP2363486, the content of which is incorporated by reference herein.

According to one embodiment of the invention, the solid tumor is at least one selected from the group consisting of
a) sarcoma,
b) blastoma, and/or
c) carcinoma.

According to one embodiment of the invention, said solid tumor is resistant to a binding moiety-toxin conjugate whose amatoxin does not comprise an —OR4 substituent at 6′-position of the indol.

According to one embodiment of the invention, the antibody or fragment or derivative thereof comprises an engineered cysteine residue.

In one embodiment, the linker is conjugated to the free SH group of said cysteine. Because cysteine is artificially introduced into the amino acid sequence of the antibody, use thereof as a conjugation site does not affect the formation of intra- and interchain disulfide bridges of the antibody, hence leaving its stability unaffected.

Preferably, such cysteine is introduced into the constant domain of the antibody, to not affect target binding of the latter. The principles of this conjugation method, are disclosed in Junutula et al (2008), the content of which is incorporated herein by reference.

According to one embodiment of the invention said cysteine residue is selected from the group consisting of heavy chain 118Cys, heavy chain 239Cys, and heavy chain 265Cys, according to the EU numbering system according to Edelman et al., Proc. Natl. Acad. Sci. USA; 63 (1969) 78-85.

The antibodies used in the present experiments comprise a D265C substitution in both Fc domains, in order to provide a cysteine residue that has such free SH groups. The respective technology is disclosed in WO2016142049 A1 assigned to the present applicant, the content of which is incorporated herein by reference, and delivers a homogenous product with a fixed drug to antibody ration (“DAR”) of 2 and a site specific conjugation.

The inventors have further shown that using D265C as a conjugation has the additional effect that, because D265 is involved in in the binding interaction of the antibody Fc region to one or more Fc[gamma] receptors, resulting in impaired binding to one or more Fc[gamma] receptors. Such antibody, or antibody amatoxin conjugate, may exhibit reduced immunomodulatory responses (like ADCC, ADCP and/or CDC responses, which are evoked by antibody binding to Fc[gamma] receptors) in the subject, and can hence contribute to reduce unwanted side effects.

According to one embodiment of the invention, the conjugate is provided for use in the treatment of a human or animal subject being (i) diagnosed for, (ii) suffering from or (iii) being at risk of developing a solid tumor, wherein said solid tumor is resistant to a binding moiety-toxin conjugate whose amatoxin does not comprise an —OR4 substituent at 6′-position of the indol.

According to one further aspect of the invention, a pharmaceutical composition for use in the treatment of a human or animal subject being (i) diagnosed for, (ii) suffering from or (iii) being at risk of developing a solid tumor is provided, which composition comprises the binding moiety-toxin conjugate as discussed above.

According to another aspect of the invention, such composition is provided for use in manufacture of a medicament for the treatment of a human or animal subject being (i) diagnosed for, (ii) suffering from or (iii) being at risk of developing a solid tumor.

According to another aspect of the invention, a method for treating or preventing a solid tumor is provided, said method comprising administering to a subject in need thereof an effective amount of such composition.

### EXAMPLES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the described invention, from a study of the drawings, the disclosure, and the appended embodiments. In the embodiments, the word “comprising” does not exclude other elements or steps, and the indefinite article “a” or “an” does not exclude a plurality. The mere fact that certain measures are recited in mutually different dependent embodiments does not indicate that a combination of these measures cannot be used to advantage. Any reference signs in the embodiments should not be construed as limiting the scope.

All amino acid sequences disclosed herein are shown from N-terminus to C-terminus; all nucleic acid sequences disclosed herein are shown 5′->3′.

### Materials and Methods

### 1. Amatoxin Linker Conjugates

In the experiments described herein, four targeted amatoxin linker conjugates are used which fall under the scope of the invention, meaning that they have a 6-OH Trp residue, either unconjugated or conjugated to a linker, as referred to under R4 in formula (i).

Further, two targeted amatoxin linker conjugates are used which do not fall under the scope of the invention because they comprise a Trp lacking the 6-OR substituent. These molecules are being used as a benchmark to demonstrate the potency increase the described constructs and conjugates in the treatment of solid tumors.

The six amatoxin linker conjugates used in the present studies are shown in the following:

| | | | |
|---|---|---|---|
| 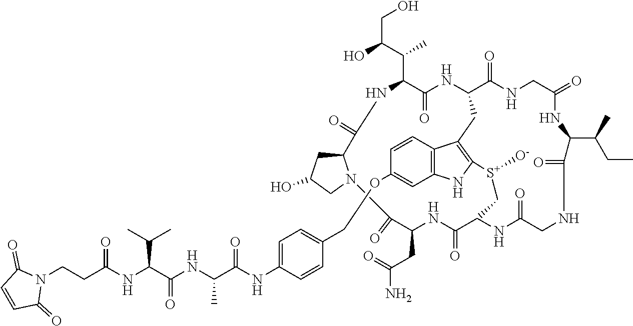 | HDP 30.1699 | 6-OH Trp Sulfoxide Linker at aa4 | according to invention |
| 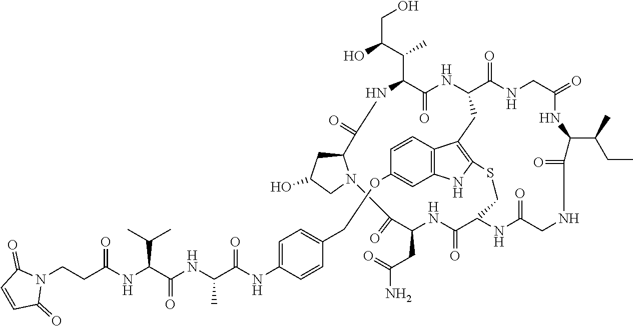 | HDP 30.2371 | 6-OH Trp thioether Linker at aa4 | according to invention |
| 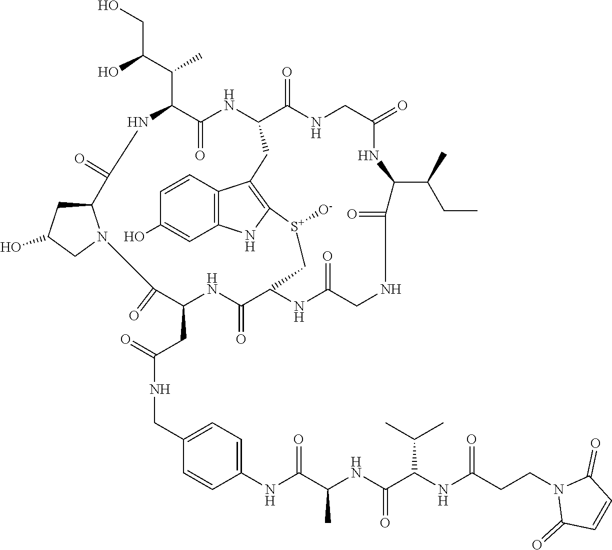 | HDP 30.2060 | 6-OH Trp Sulfoxide Linker at aa1 | according to invention |
| 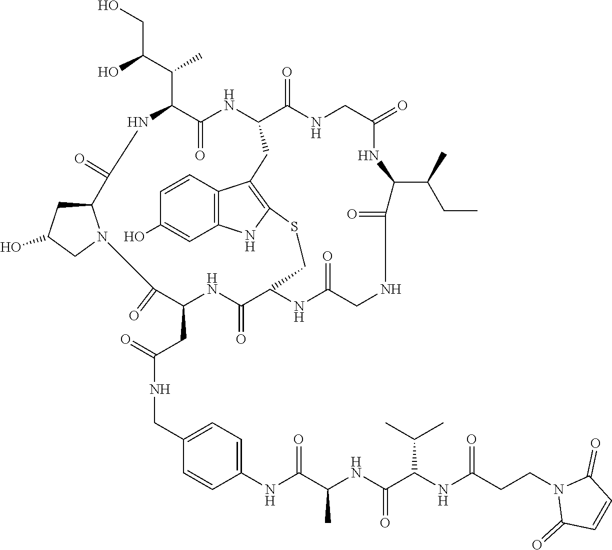 | HDP 30.2347 | 6-OH Trp thioether Linker at aa1 | according to invention |
| 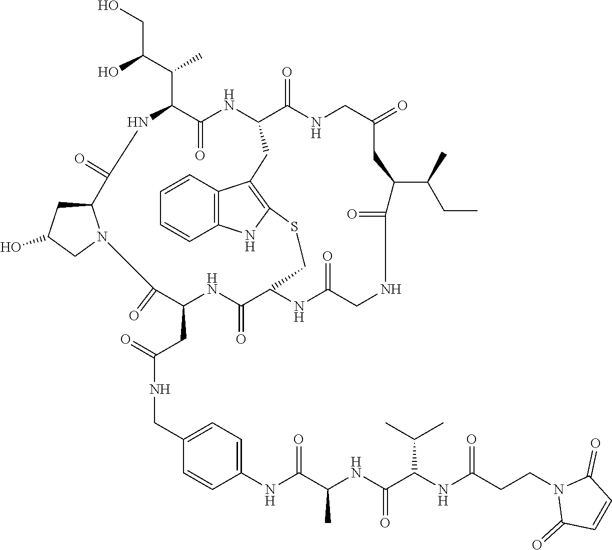 | HDP 30.2115 | Trp thioether Linker at aa1 | not according to invention |
| 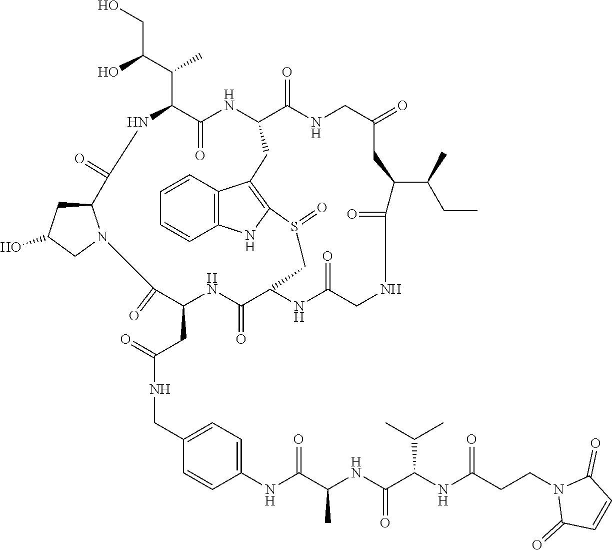 | HDP 30.2183 | Trp sulfoxide Linker at aa1 | not according to invention |

### 2. Antibodies

The following antibodies were conjugated to the amatoxin linker conjugates

| antibody | target | Format | definition | modification |
|---|---|---|---|---|
| Trastuzumab | Her-1 | IgG | drugbank entry: | D265C substitution in |
| | | | DB00072 | both Fc domain. |
| h3/F11 | PSMA | IgG | CDR sequences | D265C substitution in |
| | | | specified in | both Fc domain. |
| | | | EP2363486 | |
| chiBCE19 | CD19 | IgG | | D265C substitution in |
| | | | | both Fc domain |

Trastuzumab and h3/F11 have been described above already. chiBCE19 is enablingly disclosed in Luttgau et al 2013.

### 3. The Conjugation Technology

Antibodies were conjugated to the amatoxin linker conjugates by means of the so-called Thiomab technology. In this approach, the conjugation takes place by conjugation of the maleimide residue of the toxin linker construct to the free SH group of a cysteine residue in the antibody, as shown in the following reaction scheme:

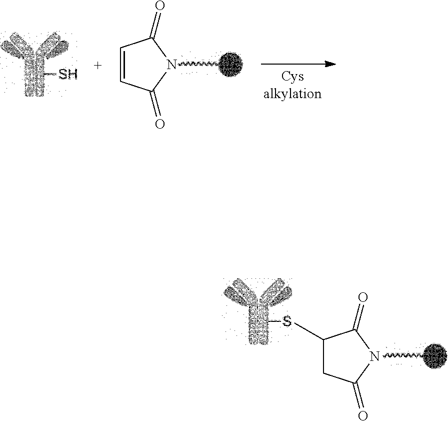

The principles of this conjugation method, are disclosed in Junutula et al (2008), the content of which is incorporated herein by reference.

The antibodies used in the present experiments comprise a D265C substitution in both Fc domains, in order to provide a cystein residue that has such free SH group. The respective technology is disclosed in WO2016142049 A1 assigned to the present applicant, the content of which is incorporated herein by reference, and delivers a homogenous product with a fixed drug to antibody ration (“DAR”) of 2 and a site specific conjugation.

### 3. Assays

### 3.1. Cell Viability Assay

Cytotoxic activity of anti PSMA and anti-Her2 amatoxin conjugates was evaluated in vitro with target-positive tumor cell lines and the chemiluminescent BrdU incorporation assay (Roche Diagnostics). Cell viability was determined after 96 h incubation with different concentrations of conjugates at 37° C. and 5% CO2 by measurement of fixed and permeabilized cells with an anti-BrdU-HRP antibody in a BMG Labtech Optima microplate reader. EC50 value of dose-response curve was calculated by Graphpad Prism 4.0 software.

Cytotoxic activity of anti CD19 amatoxin conjugates on target positive cells was assessed by CellTiter-Glo 2.0 Luminescent Cell Viability Assay (Promega, Madison, WI) in 96-well tissue culture plates. Cell viability was determined after 96 h incubation with different concentrations of conjugates at 37° C. and 5% CO2. Data analysis was performed with GraphPad Prism 7 (GraphPad Software, Inc, La Jolla, CA) software to plot curve fits and perform statistical analyses. Nonlinear variable slope curve-fits were plotted as log[ADC] vs. Response and EC50 values were generated.

### 3.2. Xenografts/Tumor Volume

LnCap xenograft model: Male CB17 SCID mice were inoculated with 2.5×10⁶ LNCap prostate carcinoma cells per mouse subcutaneously in the right flank. At a mean tumor vol. of
˜150 mm³, animals were allocated to groups on day 0. On the same day the animals received a single intravenous dose of amanitin based anti-PSMA antibody drug conjugates (ADCs) Tumor volume and body weight were determined twice per week.

Jimt-1 xenograft model: Female NMRI nude mice were inoculated with 5×10⁶ Jimt-1 breast cancer cells per mouse subcutaneously in the right flank. At a mean tumor vol. of ˜120 mm³, animals were allocated to groups on day 0. On the same day the animals received a single intravenous dose of amanitin based anti-Her2 antibody drug conjugates (ADCs) Tumor volume and body weight were determined twice per week.

Raji xenograft model: Female CB17 SCID mice were inoculated with 2.5×10⁶ Raji Burkitt's lymphoma cells per mouse subcutaneously in the right flank. At a mean tumor vol. of ˜80 mm³, animals were allocated to groups on day 0. On the same day the animals received a single intravenous dose of amanitin based anti-CD19 antibody drug conjugates (ADCs) Tumor volume and body weight were determined twice per week.

Tumor growth was monitored by caliper measurement. Tumor size was calculated according to the equation: volume=W²×L×0.5 (L=length and W=perpendicular width of the tumor, L>W).

### 3.3. Xenografts/Survival

Intravenous Nalm-6 xenograft model: Female CB17 SCID mice were inoculated with 2.5×10⁶ Raji Burkitt's lymphoma cells per mouse into the tail vein. Animals were allocated to groups on day 0. On day 3 after tumor cell injection, the animals received a single intravenous dose of amanitin based anti-CD19 antibody drug conjugates (ADCs). Body weight was determined twice per week. Survival was monitored daily.

Intravenous Raji xenograft model: Female CB17 SCID mice were inoculated with 5×10⁶ Nalm-6 B Cell precursor leukemia cells per mouse into the tail vein. Animals were allocated to groups on day 0. On day 3 after tumor cell injection, the animals received a single intravenous dose of amanitin based anti-CD19 antibody drug conjugates (ADCs). Body weight was determined twice per week. Survival was monitored daily.

3.4. Synthesis of Conjugate chiBCE19-D265C-30.2115
3.4.1. Conjugation of HDP 30.2060 to 10 mg chiBCE19-D265C

10 mg Thiomab chiBCE19-D265C in PBS buffer will be used for conjugation to HDP 30.2060.
Adjust antibody solution to 1 mM EDTA:
   2 ml antibody solution (10.0 mg)+20 μl 100 mM EDTA, pH 8.0
   Amount antibody: 10 mg=6.8×10⁻⁸ mol
      3.4.2. Uncapping of Cysteines by Reaction of Antibody with 40 Eq. TCEP:
2 ml antibody solution (6.8×10⁻⁸ mol)+54.5 μl 50 mM TCEP solution (2.72×10⁻⁶ mol)
Incubate for 3 h at 37° C. on a shaker.
Two consecutive dialyses at 4° C. in 2.0 I 1×PBS, 1 mM EDTA, pH 7.4 in a Slide-A-Lyzer Dialysis Cassette 20'000 MWCO, first dialysis ca. 4 h, second dialysis overnight
Concentrate to ca. 4.0 ml using Amicon Ultra Centrifugal Filters 50'000 MWCO.
   3.4.3. Oxidation by Reaction of Antibody with 20 Eq. Dehydroascorbic Acid (dhAA):
ca. 2 ml antibody solution (6.8×10⁻⁸ mol)+27.2 μl fresh 50 mM dhAA solution (1.36×10⁻⁶ mol)
Incubate for 3 h at RT on a shaker.
   3.4.4. Conjugation with Amanitin Using 6 Eq. HDP 30.2060 and Quenching with 25 Eq. N-Acetyl-L-Cysteine:
Solubilize 0.7 mg HDP 30.2060 in 70 μl DMSO=10 μg/μl
   ca. 2 ml antibody solution (=9.5 mg; 6.46×10⁻⁸ mol)+50.9 μl HDP 30.2060 (=509 g; 3.88×10⁻⁷ mol).
   Incubate 1 h at RT.
   Quench by addition of 16 μl 100 mM N-acetyl-L-cysteine (1.62×10⁻⁶ mol).
   Incubate 15 min at RT (or overnight at 4° C.).
   Purify each reaction mix with PD-10 columns equilibrated with 1×PBS, pH 7.4. Identify protein-containing fractions with Bradford reagent on parafilm and bring protein-containing fractions together.
   Dialysis of each antibody solution at 4° C. overnight in 2.0 I PBS, pH 7.4 and Slide-A-Lyzer Dialysis Cassettes 20'000 MWCO.
      3.4.5. Determination of Protein Concentration and Drug-Antibody Ratio (DAR) by UV-Spectra (Absorption at 280 nm and 310 nm) Using Naked Antibody Vs. ADC Adjusted to Identical Protein Concentrations.
      3.4.6. Adjust Protein Concentration to 5.0 mg/ml (3.4×10⁻⁵ M) and Bring to Sterile Conditions by Filtration. Store at 4° C.

### 4. Cell Lines

The following cell lines were used

| Her2 positive | PSMA positive | CD19 positive |
|---|---|---|
| JIMT-1 | LNCaP | Raji |
| SKBR-3 | 22RV1 | RajiLuc |
| BT474 | MDA-PCa2b | Nalm-6 |
| NCI-N87 | C4.2 | MEC-2 |

The cell lines used are derived from solid tumors, with the exception of the Raji, Nalm-6 and MEC-2 cell lines, whereas Raji is the first continuous human cell line from hematopoietic origin. It is extremely important to understand that Raji cells, which are CD19 positive, when injected into mice i.v., develop a leukemia phenotype, while when injected into mice s.c., develop a solid tumor phenotype.

### Results

### 1. Toxin-Linker Constructs Conjugated to Trastuzumab

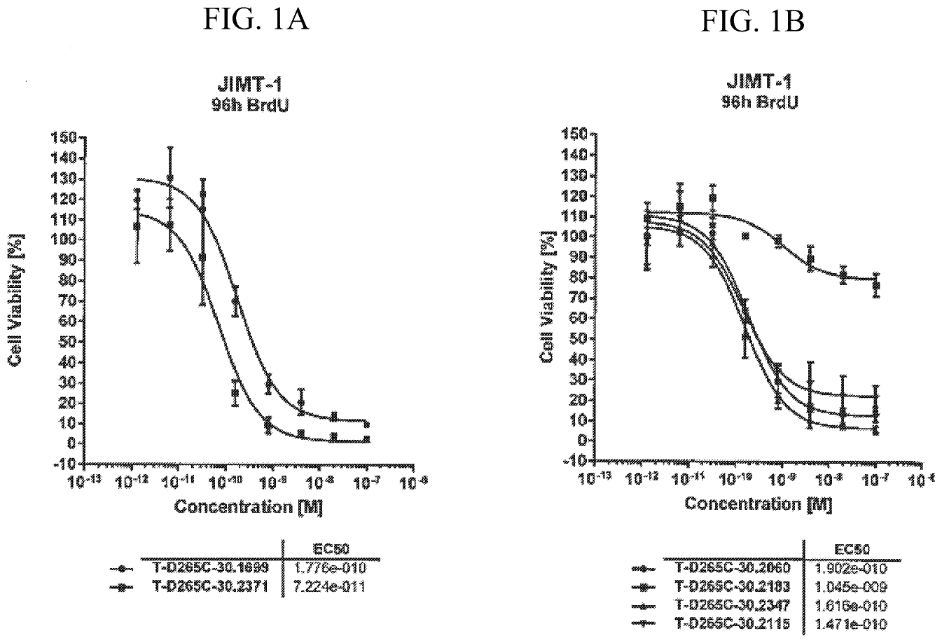

A cell viability assay was used with JIMT-1 cells treated with the toxin-linker constructs discussed above, conjugated to Trastuzumab (drugbank entry: DB00072), which had a D265C substitution in the Fc domain. JIMT-1 is a solid cancer cell line derived from a breast ductal adenocarcinoma. Results are shown in FIG. 1A and FIG. 1B.

The 6′-Hydroxy-Trp variants (where R4 of formula (i) is H or a linker which carries a reactive group Y for linking said amatoxin to a target-binding moiety) 30.1699, 30.2371, 30.2060 and 30.2347 show comparable potency, while the Trp variant 30.2115 shows a slightly decreased potency, and the Trp variant 30.2183 shows a strongly decreased potency.

The experiments shown in FIG. 1A and FIG. 1B were repeated with three other cancer cell lines derived from solid tumors, all expressing different degrees of HER2 (SKBR-3, BT474 and NCI-N87). Almost the same pattern was observed over all cell lines.

In all cases, the 6′-Hydroxy-Trp variants (where R4 of formula (i) is H or a linker which carries a reactive group Y for linking said amatoxin to a target-binding moiety) showed higher cytotoxic potential than the Trp variants (where position 6 in the Trp residue of the amanitin is substituted with a H residue).

TABLE 2
EC50values [M] of the conjugates on the different cell lines

| Conjugate | Characteristics | JIMT-1 | SKBR-3 | BT474 | NCI-N87 |
|---|---|---|---|---|---|
| T-D265C- | SO; 6-OH-W; | 1.8 × | 9.9 × 10⁻¹² | 6.8 × 10⁻¹¹ | 1.2 × |
| 30.1699 | AA4 | 10⁻¹⁰ | | | 10⁻¹¹ |
| T-D265C- | S; 6-OH-W; | 7.2 × | 1.6 × | 4.6 × | 9.5 × |
| 30.2371 | AA4 | [symbol] | [symbol] | [symbol] | [symbol] |
| T-D265C- | SO; 6-OH-W; | 1.9 × | 2.6 × 10⁻¹¹ | 1.1 × 10⁻¹⁰ | 1.6 × |
| 30.2060 | AA1 | 10⁻¹⁰ | | | 10⁻¹¹ |
| | | (10%) | | | |
| T-D265C- | S; 6-OH-W; | 1.6 × | 7.9 × 10⁻¹² | 6.4 × 10⁻¹¹ | 3.7 × |
| 30.2347 | AA1 | 10⁻¹⁰ | | | 10⁻¹¹ |
| T-D265C- | S; W; AA1 | 1.5 × | 5.8 × 10⁻¹² | 5.6 × 10⁻¹¹ | 5.1 × |
| 30.2115 | | 10⁻¹⁰ | | | 10⁻¹¹ |
| | | (25%) | | | |
| T-D265C- | SO; W; AA1 | nfb | 4.2 × | 4.2 × | 2.4 × |
| 30.2183 | | | [symbol] | [symbol] | [symbol] |

Bold print: highest cytotoxic potential;
italics: lowest cytotoxic potential.
“SO” means R₅ according to the amanitin of formula (i) = O,
“S” means R₅ according to the amanitin of formula (i) is absent,
“6-OH-W” refers to the embodiment shown in formula (i) with OR₄,
“W” refers to an embodiment which is not in accordance with the invention where position 6 in the Trp residue of the amanitin is substituted with a H residue, and “AA4/AA1” mean the Amino Acid residue in the amanitin to which the linker is conjugated.
“Ngh” means: no full-blown cytotoxic potential with >50% residual cell viability

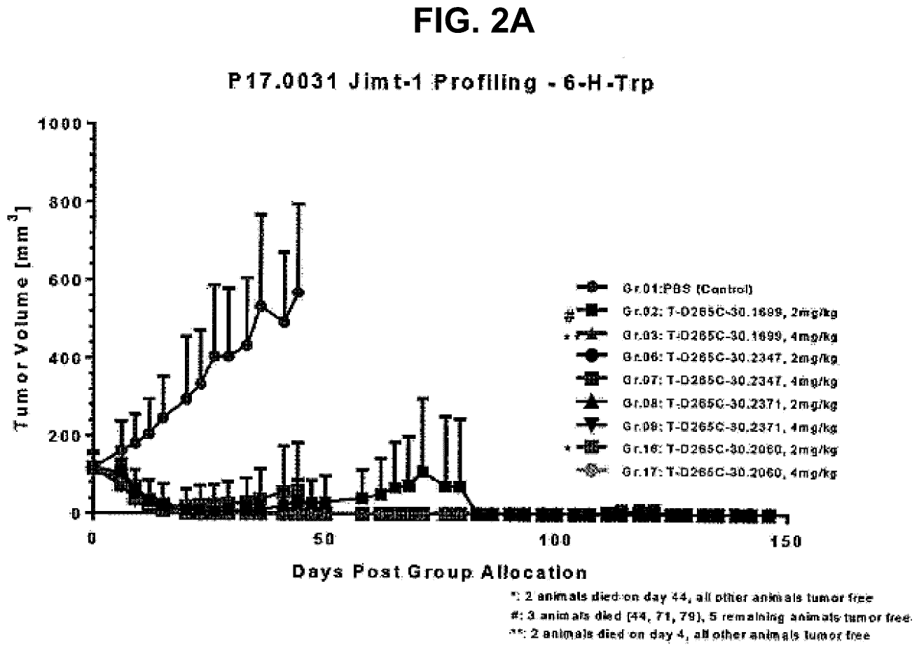

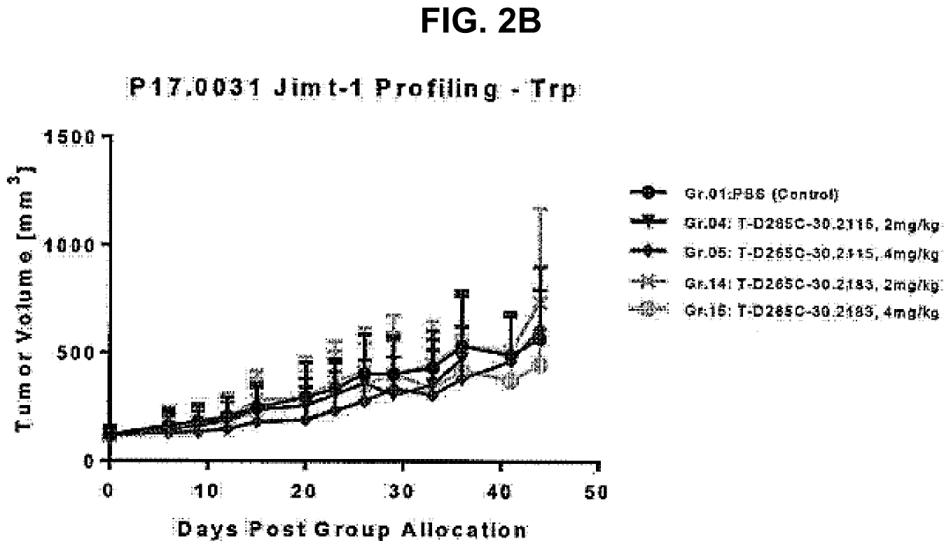

Growth inhibition experiments were then carried out with tumor xenografts of JIMT cells, which were then treated with the toxin-linker constructs discussed above, conjugated to Trastuzumab (drugbank entry: DB00072), which had a D265C substitution in the Fc domain. Results are shown in FIG. 2.

The 6′-Hydroxy-Trp variants (where R4 of formula (i) is H or a linker which carries a reactive group Y for linking said amatoxin to a target-binding moiety) 30.1699, 30.2371, 30.2060 and 30.2347 show strong tumor shrinking activity (FIG. 2A), while the Trp variants 30.2115 and 30.2183 show a significantly decreased tumor shrinking activity (FIG. 2B).

### 2. Toxin-Linker Constructs Conjugated to an Anti-PSMA Antibody

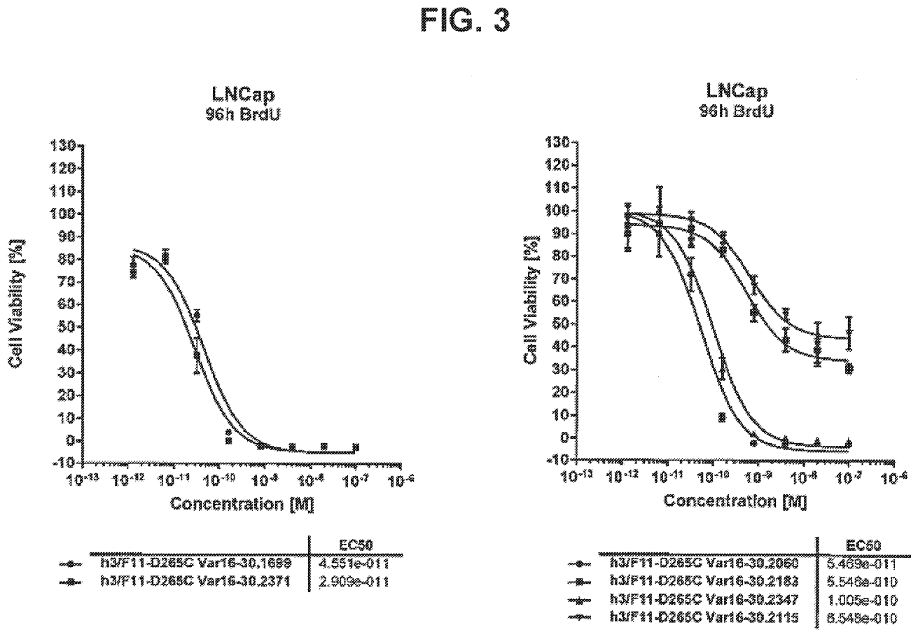

A cell viability assay was used with LNCaP cells treated with the toxin-linker constructs discussed above, conjugated to the anti-PSMA antibody h3/F11, which had a D265C substitution in the Fc domain. LNCaP is a solid prostate adenocarcinoma cell line derived from a left supraclavicular lymph node metastasis. Results are shown in FIG. 3

The 6′-Hydroxy-Trp variants (where R4 of formula (i) is H or a linker which carries a reactive group Y for linking said amatoxin to a target-binding moiety) 30.1699, 30.2371, 30.2060 and 30.2347 show comparable potency, while the Trp variants 30.2115 and 30.2183 show a strongly decreased potency.

The experiments shown in FIG. 3 were repeated with three other solid cancer cell lines, all expressing different degrees of PSMA (22RV1, MDA-PCa2b, and C4.2). Almost the same pattern was observed over all cell lines.

In all cases, the 6′-Hydroxy-Trp variants (where R4 of formula (i) is H or a linker which carries a reactive group Y for linking said amatoxin to a target-binding moiety) slightly higher cytotoxic potential than the Trp variants (where position 6 in the Trp residue of the amanitin is substituted with a H residue).

TABLE 3
EC50values [M] of the conjugates on the different cell lines

| Conjugate | Characteristics | LNCaP | 22RV1 | MDA-PCa2b | C4.2 |
|---|---|---|---|---|---|
| h3/F11- | SO; 6-OH- | 4.6 × | 1.1 × 10⁻¹⁰ | 1.6 × 10⁻¹⁰ | 4.3 × |
| D265C | W; AA4 | 10⁻¹¹ | | | 10⁻¹¹ |
| 30.1699 | | | | | |
| h3/F11- | S; 6-OH- | 2.9 × | 3.9 × | 2.6 × | 3.9 × |
| 1D265C | W; AA4 | [symbol] | [symbol] | [symbol] | [symbol] |
| 30.2371 | | | | | |
| h3/F11- | SO; 6-OH- | 5.5 × | 2.4 × 10⁻¹⁰ | 8.5 × 10⁻¹¹ | 6.8 × |
| 1D265C | W; AA1 | 10⁻¹¹ | (15%) | | 10⁻¹¹ |
| 30.2060 | | | | | |
| h3/F11- | S; 6-OH-W; | 1.0 × | 8.2 × 10⁻¹¹ | 4.5 × 10⁻¹¹ | 8.2 × |
| 1D265C | AA1 | 10⁻¹⁰ | (10%) | | 10⁻¹¹ |
| Var16- | | | | | |
| 30.2347 | | | | | |
| h3/F11- | S; W; AA1 | 6.5 × | 1.5 × 10⁻¹⁰ | 6.3 × 10⁻¹¹ | 5.6 × |
| 1D265C | | [symbol] | (25%) | | 10⁻¹⁰ |
| Var16- | | (50%) | | | (25%) |
| 30.2115 | | | | | |
| h3/F11- | SO; W; AA1 | 5.5 × | 3.6 × [symbol] | 3.6 × [symbol] | nfb |
| 1D265C | | 10⁻¹⁰ | (35%) | | |
| Var16- | | (40%) | | | |
| 30.2183 | | | | | |

Bold print: highest cytotoxic potential;
italics: lowest cytotoxic potential.
“SO” means R₅ according to the amanitin of formula (i) = 0,
“S” means R₅ according to the amanitin of formula (i) is absent,
“6-OH-W” refers to the embodiment shown in formula (i) with OR₄,
“W” refers to an embodiment which is not in accordance with the invention where position 6 in the Trp residue of the amanitin is substituted with a H residue, and
“AA4/AA1” mean the Amino Acid residue in the amanitin to which the linker is conjugated.
“Nfb” means: no full-blown cytotoxic potential with >50% residual cell viability.
(X %) means residual viability.

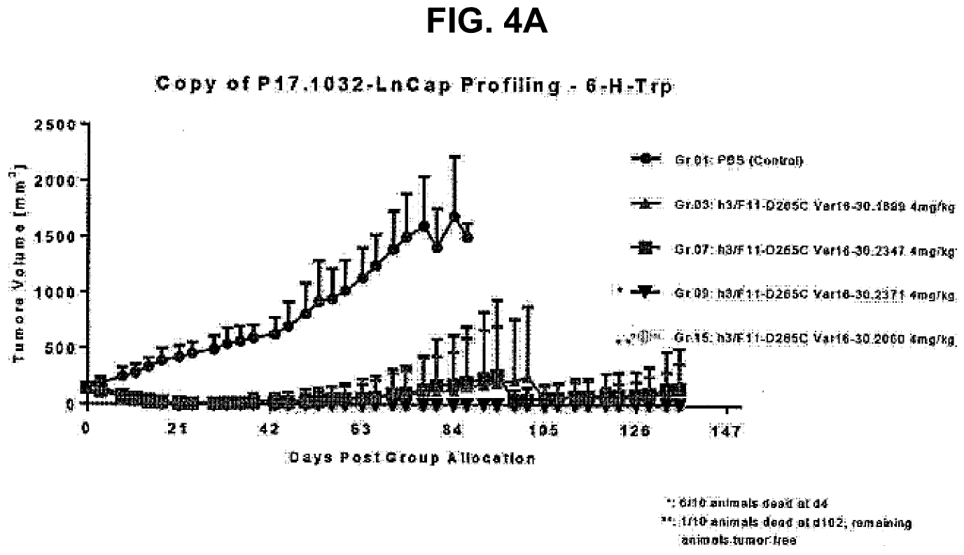

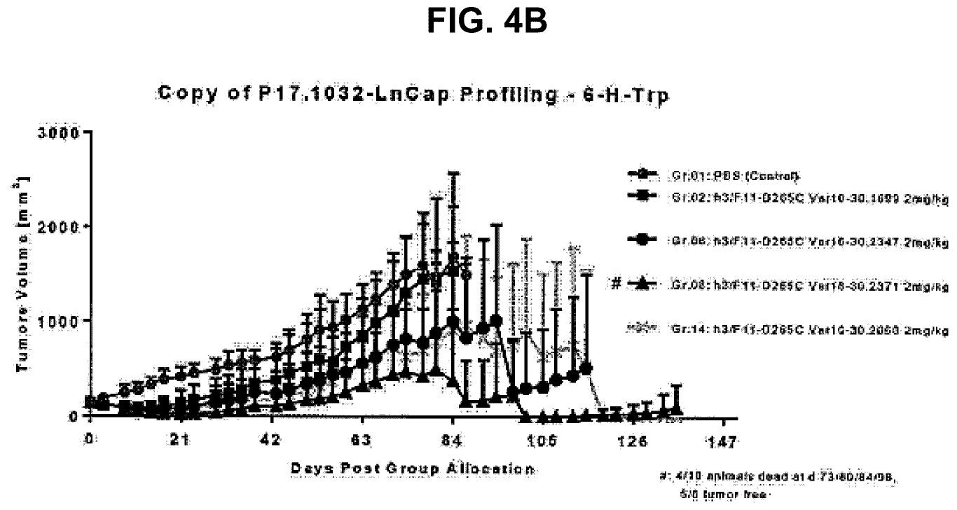

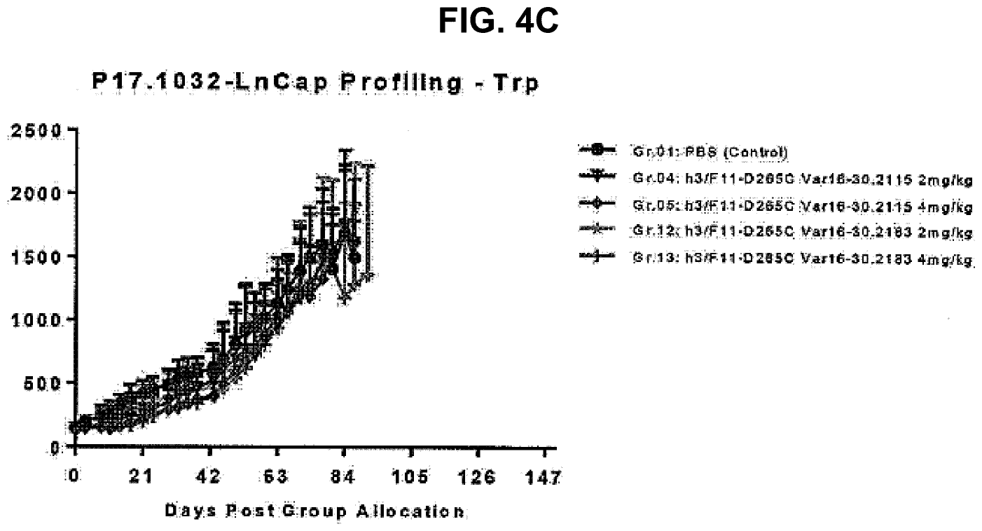

Growth inhibition experiments were then carried out with tumor xenografts of LnCap cells, which were treated with the toxin-linker constructs discussed above, conjugated to the anti-PSMA antibody h3/F1, which had a D265C substitution in the Fc domain. Results are shown in FIG. 4.

The 6′-Hydroxy-Trp variants (where R4 of formula (i) is H or a linker which carries a reactive group Y for linking said amatoxin to a target-binding moiety) 30.1699, 30.2371, 30.2060 and 30.2347 show strong tumor shrinking activity (FIG. 4A, B), while the Trp variants 30.2115 and 30.2183 show a significantly decreased tumor shrinking activity (FIG. 4C).

3. Toxin-Linker Constructs Conjugated to an antiCD19 Antibody

A cell viability assay was used with Raji cells treated with the toxin-linker constructs discussed above, conjugated to the antiCD19 antibody chiBCE19, which had a D265C substitution in the Fc domain. Raji is a cell line derived from Burkitt's lymphoma.

In this case both, the 6′-Hydroxy-Trp variants (where R4 of formula (i) is H or a linker which carries a reactive group Y for linking said amatoxin to a target-binding moiety) 30.1699, 30.2371, 30.2060 and 30.2347 and the Trp variants 30.2115 and 30.2183 show comparable potency.

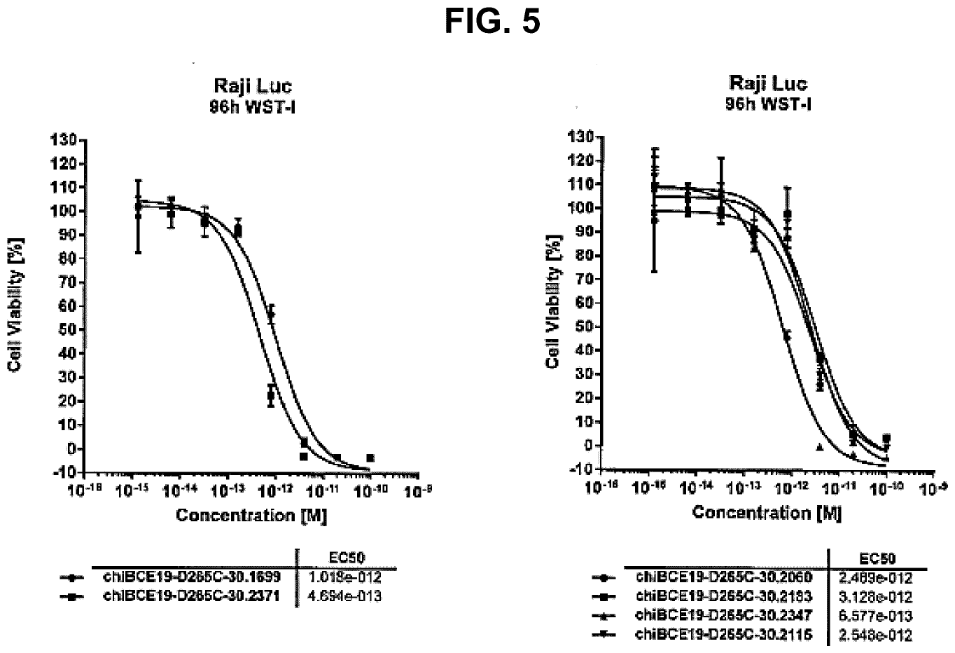

The experiments shown in FIG. 5 were repeated with two other cancer cell lines, all expressing different degrees of CD19 (Raji-Luc and MEC-2). Almost the same pattern was observed over all cell lines.

In all cases, the 6′-Hydroxy-Trp variants (where R4 of formula (i) is H or a linker which carries a reactive group Y for linking said amatoxin to a target-binding moiety) shows comparable cytotoxic potential as the Trp variants (where position 6 in the Trp residue of the amanitin is substituted with a H residue).

TABLE 4
EC50values [M] of the conjugates on the different cell lines

| Conjugate | Characteristics | Raji | Raji-Luc | MEC-2 |
|---|---|---|---|---|
| chiBCE19- | SO; 6-OH- | 2.9 × 10⁻¹² | 1.0 × 10⁻¹² | 1.1 × |
| D265C- | W; AA4 | | | 10⁻¹¹ |
| 30.1699 | | | | |
| chiBCE19- | S; 6-OH- | 1.9 × | 4.7 × | 2.9 × |
| D265C- | W; AA4 | [symbol] | [symbol] | [symbol] |
| 30.2371 | | | | |
| chiBCE19- | SO; 6-OH- | 2.5 × 10⁻¹¹ | 2.5 × 10⁻¹² | 3.4 × |
| D265C- | W; AA1 | | | 10⁻¹¹ |
| 30.2060 | | | | (20%) |
| chiBCE19- | S; 6-OH- | 8.2 × 10⁻¹² | 6.6 × 10⁻¹³ | 1.6 × |
| D265C- | W; AA1 | | | 10⁻¹¹ |
| 30.2347 | | | | (10%) |
| chiBCE19- | S; W; AA1 | 4.2 × 10⁻¹¹ | 2.5 × 10⁻¹² | 2.7 × |
| D265C- | | | | 10⁻¹¹ |
| 30.2115 | | | | (20%) |
| chiBCE19- | SO; W; AA1 | 2.6 × [symbol] | 3.1 × | 8.6 × |
| D265C- | | (30%) | [symbol] | [symbol] |
| 30.2183 | | | | (30%) |

Bold print highest cytotoxic potential;
italics: lowest cytotoxic potential.
“SO” means R₅ according to the amanitin of formula (i) = of formula (i) is 0,
“S” means R₅ according to the amanitin absent,
“6-OH-W” refers to formula (i) with OR₄,
“W” refers to an the embodiment shown in embodiment which is not in accordance with the invention where position 6 in the Trp residue of the amanitin is substituted with a H residue, and
“AA4/AA1” mean the Amino Acid residue in the amanitin to which the linker is conjugated.
“Nfb” means: no full-blown cytotoxic potential with >50% residual cell viability.
(X %) means residual viability.

Survival rates were then investigated with tumor xenografts of Raji cells (with or without transfected luciferase) or Nalm-6 cells obtained by i.v. administration of the respective cells.

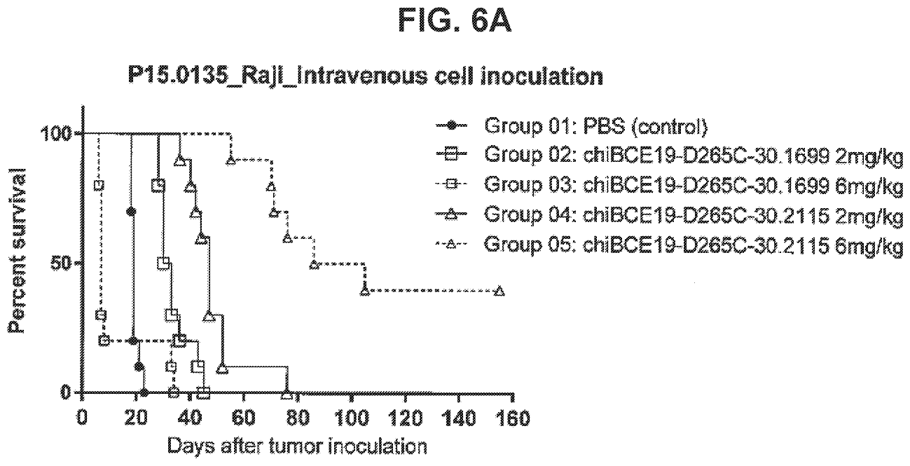

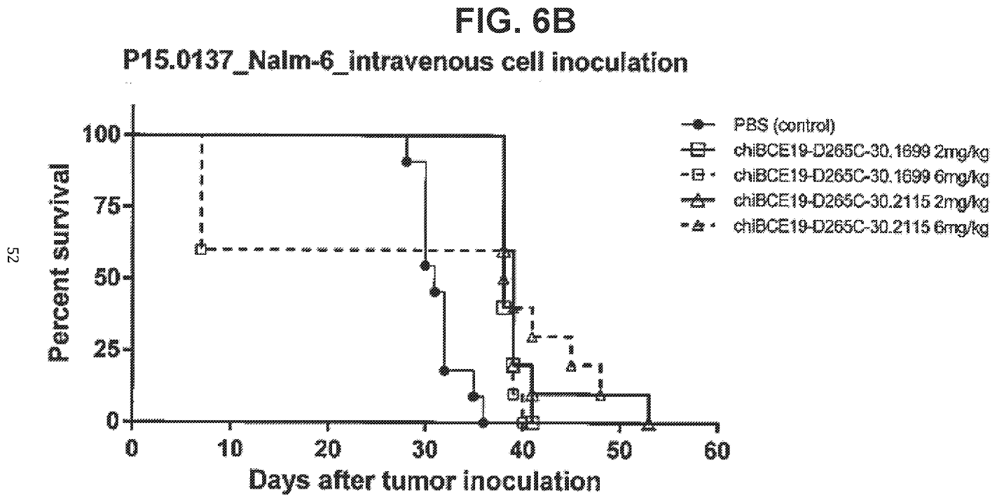

The mice were then treated with a single i.v. dose of the toxin-linker constructs discussed above, conjugated to the antiCD19 antibody chiBCE19, which had a D265C substitution in the Fc domain. Raji is a cell line derived from Burkitt's lymphoma, Nalm-6 is a cell line derived from acute lymphoblastic leukemia (ALL). Results are shown in the Kaplan Meier curves of FIG. 6.

When administered i.v., Raji as well as Nalm-6 cells develop a phenotype that mimics that of a leukemia, i.e., a liquid tumor, while, when administered s.c., only Raji cells develop a phenotype that mimics that of a solid tumor, while Nalm-6 cells still develop a liquid tumor.

Survival experiments were carried out with i.v. injected Raji Luc (FIG. 6A) or NALM6 (FIG. 6B) cells. In all cases, it turned out that the variant which has the 6-OH Trp (30.1699) has lower potency than the Trp variant 30.2115.

Growth inhibition experiments were then carried out with tumor xenografts of Raji cells injected into mice s.c. This approach leads to tumors that mimic the phenotype of a solid tumor. Treatment was done with the toxin-linker constructs discussed above, conjugated to the antiCD19 antibody chiBCE19, which had a D265C substitution in the Fc domain.

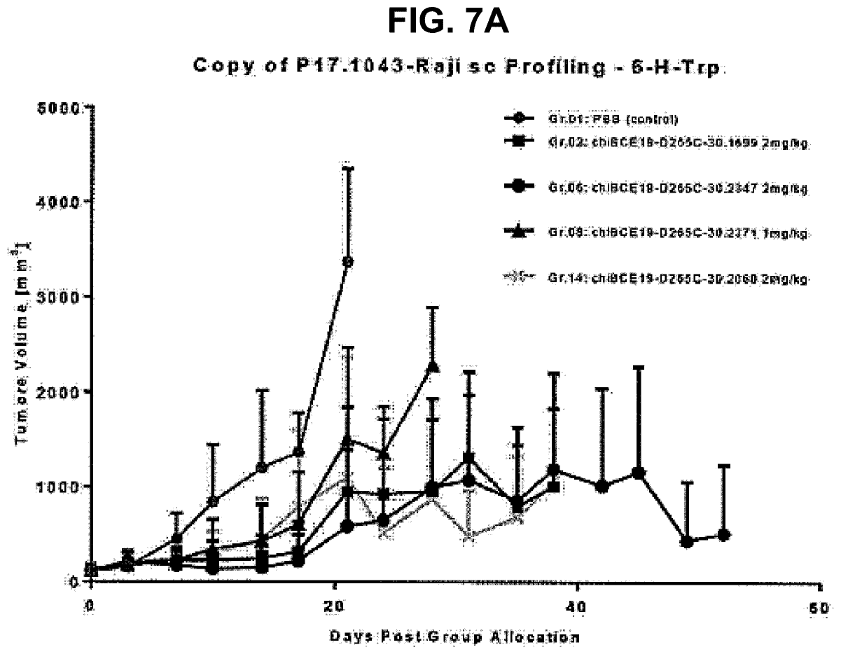

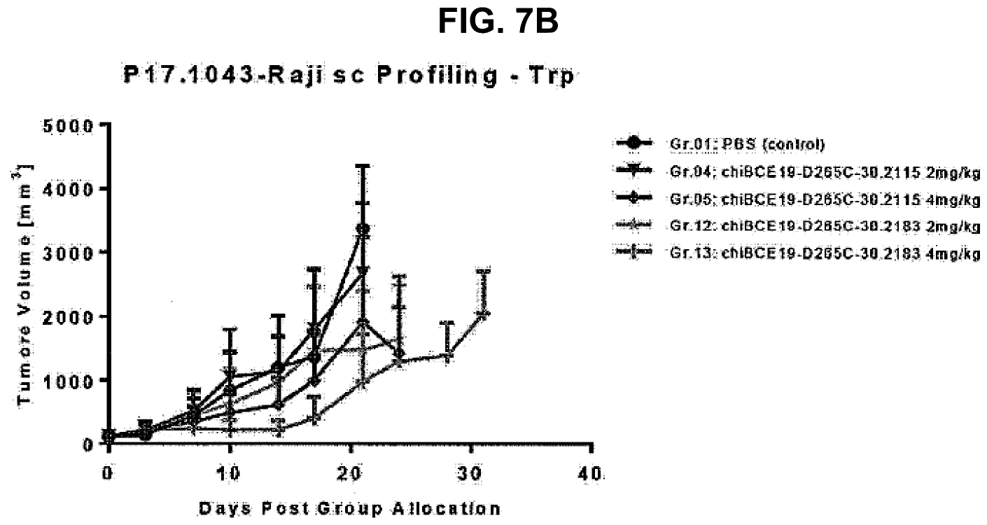

The 6′-Hydroxy-Trp variants (where R4 of formula (i) is H or a linker which carries a reactive group Y for linking said amatoxin to a target-binding moiety) 30.1699, 30.2371, 30.2060 and 30.2347 show strong tumor shrinking activity (FIG. 7A), while the Trp variants 30.2115 and 30.2183 show a significantly decreased tumor shrinking activity (FIG. 7B).

| amatoxin | | | | | Efficacy | | Efficacy | | | |
|---|---|---|---|---|---|---|---|---|---|---|
| linker | Efficacy | MTD HER2 | Efficacy | MTD PSMA | Raji-Luc | Efficacy | Nalm-6 | Efficacy | MTD CD19 | HNSTD DIG |
| Conjugate | JIMT-1 | mice mg/kg | LnCap | mice mg/kg | iv | Raji i.v. | i.v. | Raji sc | mice mg/kg | Cyno mg/kg |
| 30.1699 | ++ | 2 | + | 4 | + | + | + | ∘ | 4 | >3 |
| 30.2371 | ++ | 4 | ++ | 2 | ++ | −− | −− | ∘ | 2 | >2 |
| 30.2060 | ++ | >10 | + | >10 | + | −− | −− | ∘ | >10 | <7.5 |
| 30.2347 | ++ | 4 | + | 4 | ++ | −− | −− | ∘ | 6 | <5 |
| 30.2115* | −− | 6 | −− | >10 | + | ++ | + | −− | >10 | <7.5 |
| 30.2183* | −− | >10 | −− | >10 | + | −− | −− | −− | >10 | |

*Amatoxin linker constructs not according to the invention.
MTD: maximum tolerated Dose,
HNSTD: highest non-severely toxic dose,
Cyno: Cynomolgus monkey,
DIG: Digoxigenin antibody which does not cross react with hosts targets

### REFERENCES

Junutula et al., Nat. Biotechnol. 26 (8), 925-932 (2008)
Lüttgau et al., Antibodies (2013) 2, 338-352

1. A method of treating a patient afflicted with a solid tumor, the method comprising:
administering to said patient an effective amount of a binding moiety-toxin conjugate,
wherein the binding moiety-toxin conjugate comprises an amatoxin-linker construct selected from the group consisting of: 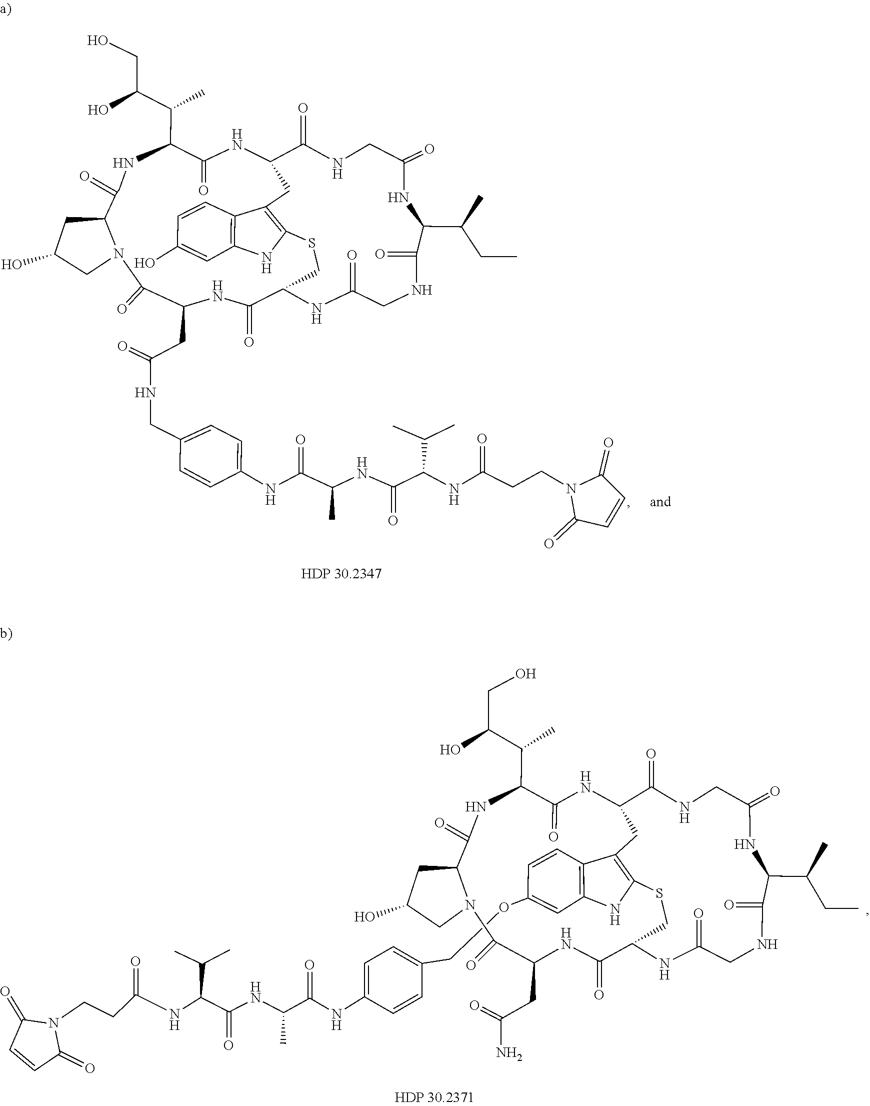
and
wherein said solid tumor is resistant to a binding moiety-toxin conjugate whose amatoxin according to formula (I) does not comprise an —OR₄ substituent at 6′-position of the indol, wherein formula (I) is defined as 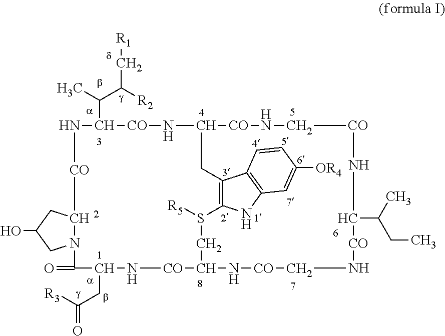
and wherein
R1 and R2 are each —OH,
R3 is NH2, or a linker which carries a reactive group Y for linking said amatoxin to a target-binding moiety,
R4 is H or a linker which carries a reactive group Y for linking said amatoxin to a target-binding moiety,
R5 is absent or =0,
wherein R3 and R4 cannot be the same, and
wherein the target-binding moiety is an antibody, antibody fragment, antibody-based binding protein, or an antibody mimetic, all of which retain target binding properties.

2. The method according to claim 1, wherein said target-binding moiety binds at least one target selected from the group consisting of Her2, and PSMA.

3. The method according to claim 1, wherein the solid tumor is at least one selected from the group consisting of (a) sarcoma, (b) blastoma, and (c) carcinoma.

4. The method according to claim 1, wherein said target-binding moiety comprises an antibody or fragment or a derivative thereof which comprises an engineered cysteine residue.

5. The method according to claim 4, wherein said cysteine residue is selected from the group consisting of heavy chain 118Cys, heavy chain 239Cys, and heavy chain 265Cys, according to the EU numbering system.
